(19) United States
(12) Patent Application Publication
(10) Pub. No.: US 2007/0134186 A1
(43) Pub. Date: Jun. 14, 2007

(54) Cosmetic composition comprising at least one oxyethylenated sorbitan ester, at least one cationic polymer and at least one solid fatty substance, and a cosmetic treatment process

(76) Inventors: Patricia Mezure, Bougival (FR); Pascale Lazzeri, Levallois Perret (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/588,284

(22) Filed: Oct. 27, 2006

Related U.S. Application Data
(60) Provisional application No. 60/736,295, filed on Nov. 15, 2005.

(30) Foreign Application Priority Data
Oct. 28, 2005 (FR) 05 53286

(51) Int. Cl. A61K 8/72; A61K 8/37

(52) U.S. Cl. 424/70.11; 424/70.31

(57) ABSTRACT

The present disclosure relates to a cosmetic composition comprising, in a cosmetically acceptable aqueous medium, at least one ester of oxyethylenated sorbitan and of a saturated or unsaturated, linear or branched C₈-C₃₀ fatty acid, with a number of moles of ethylene oxide of less than or equal to 20, at least one non-silicone cationic polymer with a cationic charge density of greater than or equal to 4 meq/g and at least one non-silicone solid fatty compound, and to a cosmetic process for treating keratin materials, for example the hair. These compositions have an improved conditioning effect, such as smoothing of the ends.

This application claims benefit of U.S. Provisional Application No. 60/736,295, filed Nov. 15, 2005, the contents of which are incorporated herein by reference. This application also claims benefit of priority under 35 U.S.C. § 119 to French Patent Application No. FR 05 53286, filed Oct. 28, 2005, the contents of which are also incorporated herein by reference.

The present disclosure relates to a cosmetic composition, such as a hair conditioner, comprising at least one cationic surfactant, at least one oxyethylenated sorbitan ester, at least one non-silicone cationic polymer and at least one non-silicone solid fatty substance, and to a cosmetic process for treating keratin materials, for instance the hair.

It is known that hair that has been sensitized (i.e., damaged and/or embrittled) to varying degrees under the action of atmospheric agents and/or under the action of mechanical or chemical treatments, such as dyeing, bleaching and/or permanent-waving, is often difficult to disentangle and/or to style, and/or lacks softness.

Cosmetic compositions comprising cationic surfactants and thickening polysaccharides, such as starch or celluloses, have been proposed for treating keratin materials, and such as the hair.

However, such compositions may have drawbacks such as rinseability problems, stability problems, difficulties in distributing them over the keratin materials and/or also insufficient cosmetic properties.

It has been recommended to use cationic polymers, cationic silicones or cationic surfactants in compositions for washing or caring for keratin materials such as the hair, to facilitate the disentangling of the hair and to give it softness and suppleness. However, the use of cationic polymers or cations for this purpose may have various drawbacks. On account of their high affinity for the hair, some of these polymers may become deposited in substantial amount during repeated use, and lead to undesirable effects such as an unpleasant, laden feel, stiffening of the hair, and/or adhesion between the fibers that affects styling.

Furthermore, the care used for very sensitized hair may be insufficient to treat the ends, which are usually very damaged.

In summary, it is found that the current conditioning cosmetic compositions are not always entirely satisfactory. Thus, it is sought to obtain cosmetic compositions that have very good cosmetic properties, for example on very sensitized hair.

The present inventors have now discovered, surprisingly, that the combination of at least one particular oxyethylenated sorbitan ester, with at least one non-silicone cationic polymer of particular cationic charge and at least one non-silicone solid fatty compound allows at least one of these drawbacks to be overcome.

Hair treated with this composition, for example, may be smooth, disentangle easily, be shiny, supple and/or individualized, and/or have a soft feel with no feeling of residues. The hair may have a natural, unladen appearance. The smoothness may be uniform from the roots to the ends, and the ends may show less splitting.

Moreover, these effects may be remanent over time.

The present inventors have discovered that the addition of the above-defined readily oxyethylenated sorbitan ester makes it possible, surprisingly, to reduce the discomfort reactions (itching, redness, etc.), such as on the scalp, of compositions comprising surfactants liable to cause reactions of this type when they are used alone.

Thus, according to the present disclosure, novel cosmetic compositions are now proposed, comprising, in a cosmetically acceptable aqueous medium, at least one ester of oxyethylenated sorbitan and of a saturated or unsaturated, linear or branched C₈-C₃₀ fatty acid, with a number of moles of ethylene oxide of less than or equal to 20, at least one non-silicone cationic polymer with a cationic charge density of greater than or equal to 4 meq/g and at least one non-silicone solid fatty substance.

Another aspect of the present disclosure is a cosmetic process for treating keratin materials, such as the hair, using the composition as disclosed herein.

Yet, another embodiment of the present disclosure relates to the use of the composition as a hair conditioner.

Other subjects, characteristics, aspects and benefits of the present disclosure will emerge more clearly upon reading the description and the various examples that follow.

As used herein, the term “sensitized hair” is understood to mean hair that has undergone external physical attack (by light, heat, waves, etc.), mechanical attack (by repeated blow-drying, combing or brushing, etc.) and/or chemical attack (by oxidation dyeing, bleaching, permanent-waving, relaxing, etc.). Among these types of attack, the deleterious nature of chemical attack is noted. In at least one embodiment, the compositions according to the present disclosure are effective on hair sensitized by chemical attack.

As used herein, the term “at least one” will be understood as meaning “one or more”, i.e., one, two, three or more.

As used herein, the term “cosmetically acceptable medium” is understood to mean a medium that is compatible with any keratin material, such as the skin, the hair, the nails, the eyelashes, the eyebrows or the lips and any other area of body and facial skin.

As used herein, the term “non-silicone fatty substance” is understood to mean any organic compound comprising in its structure at least one hydrocarbon-based chain comprising at least 10 carbon atoms, not containing any silicon atoms.

The solid fatty substances may have a melting point of greater than or equal to 35° C. and/or have a viscosity at a temperature of 40° C. and at a shear rate of 1 s⁻¹ of greater than or equal to 1 Pa·s.

In another embodiment of the present disclosure, the solid fatty substances according to the present disclosure are non-polymeric, i.e., they do not comprise any repeated monomer units other than alkylene oxide units.

The fatty acids of the sorbitan esters of a C₈-C₃₀ fatty acid, comprise a number of moles of ethylene oxide of less than or equal to 20, comprising 8 to 24 carbon atoms and, for example, from 8 to 18 carbon atoms. The fatty acids may be chosen, for instance, from lauric acid, palmitic acid, oleic acid and stearic acid, and in at least one embodiment, from lauric acid and stearic acid, such as from lauric acid.

In at least one embodiment, monoesters of a C₈-C₂₄ fatty acid and of oxyethylenated sorbitan may be used. The number of moles of ethylene oxide may be less than or equal to than 10 and, for instance, ranging from 3 to 8 mols of ethylene oxide, such as equal to 4 mol.

Non-limiting examples of the sorbitan esters are sorbitan monolaurate oxyethylenated with 4 mol of ethylene oxide (4 EO) or polysorbate 21, sorbitan monostearate oxyethylenated with 4 mol of ethylene oxide (4 EO) or polysorbate 61, and sorbitan monooleate oxyethylenated with 5 mol of ethylene oxide (5 EO) or polysorbate 81.

As noted above, Polysorbate 21 may be used and is sold, for example, under the name TWEEN 21 by the company Uniqema.

In another embodiment of the present invention, the composition may comprise mixtures of oxyethylenated sorbitan esters, such as polysorbate 21 with polysorbate 20 (sorbitan monolaurate oxyethylenated with 20 EO).

In, yet, another embodiment of the present invention, the oxyethylenated sorbitan ester may be present in the cosmetic composition in proportions ranging from 0.1% to 10% and, for example, from 0.5% to 5% by weight relative to the total weight of the composition.

The cosmetic composition as disclosed herein comprises at least one cationic polymer whose cationic charge density is greater than or equal to 4 milliequivalents per gram (meq/g), such as ranging from 4 to 25 meq/g and further, for example, from 4 to 8 meq/g.

As used herein, the cationic charge density of a polymer is understood to mean the number of moles of cationic charges per unit of mass of polymer under conditions wherein the polymer is totally ionized. It may be determined by calculation if the structure of the polymer is known, i.e., the structure of the monomers constituting the polymer and their molar or weight proportion. It may also be determined experimentally via the Kjeldahl method.

The cationic polymers with a cationic charge density of greater than or equal to 4 meq/g that may be used in accordance with the present disclosure may be chosen from all those already known per se as improving the cosmetic properties of the hair treated with compositions, such as those described in European Patent Application No. 0 337 354 and in French Patent Application Nos. 2 270 846, 2 383 660, 2 598 611, 2 470 596 and 2 519 863.

As used herein, the term “cationic polymer” is understood to mean any polymer comprising cationic groups and/or groups that may be ionized into cationic groups.

The cationic polymers may be chosen from those comprising units comprising primary, secondary, tertiary and/or quaternary amine groups that either may form part of the main polymer chain or may be borne by a side substituent directly attached thereto.

The cationic polymers used may have a number-average molecular mass ranging from 500 to 5×10⁶ and, for instance, ranging from 10³ to 3×10⁶.

Further, among the cationic polymers that may be used, non-limiting mention may be made of polymers of the polyamine, polyamino amide and polyquaternary ammonium type. These are known products.

Still further, among the polymers of the polyamine, polyamino amide and polyquaternary ammonium type that may be used in accordance with the present disclosure, non-limiting mention may be made of those described in French Patent Nos. 2 505 348 and 2 542 997. Among these polymers, non-limiting examples include:

(1) quaternized or non-quaternized vinylpyrrolidone/dialkylaminoalkyl (meth)acrylate copolymers,

(2) polymers comprising piperazinyl units and divalent alkylene or hydroxyalkylene radicals comprising straight or branched chains, optionally interrupted by oxygen, sulfur or nitrogen atoms or by aromatic or heterocyclic rings, as well as the oxidation and/or quaternization products of these polymers. Such polymers are described, for example, in French Patent Nos. 2 162 025 and 2 280 361;

(3) water-soluble polyamino amides prepared by polycondensation of an acidic compound with a polyamine; these polyamino amides may be crosslinked with an epihalohydrin, a diepoxide, a dianhydride, an unsaturated dianhydride, a bis-unsaturated derivative, a bis-halohydrin, a bis-azetidinium, a bis-haloacyidiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyamino amide; these polyamino amides may be alkylated or, if they comprise at least one tertiary amine function, they can be quaternized. Such polymers are described, for example, in French Patent Nos. 2 252 840 and 2 368 508;

(4) polyaminoamide derivatives resulting from the condensation of polyalkylene polyamines with polycarboxylic acids followed by alkylation with difunctional agents. Non-limiting mention may be made, for example, of adipic acid/dialkylaminohydroxyalkyl-dialkylenetriamine polymers wherein the alkyl group comprises from 1 to 4 carbon atoms and is chosen from, for example, methyl, ethyl or propyl. Such polymers are described, for example, in French Patent No. 1 583 363.

(5) polymers obtained by reaction of a polyalkylene polyamine comprising two primary amine groups and at least one secondary amine group with a dicarboxylic acid chosen from diglycolic acid and saturated aliphatic dicarboxylic acids having from 3 to 8 carbon atoms. The molar ratio between the polyalkylene polyamine and the dicarboxylic acid ranges from 0.8:1 to 1.4:1; the polyamino amide resulting therefrom being reacted with epichlorohydrin in a molar ratio of epichlorohydrin relative to the secondary amine group of the polyamino amide ranges from 0.5:1 to 1.8:1. Such polymers are described, for example, in U.S. Pat. Nos. 3,227,615 and 2,961,347.

(6) cyclopolymers of alkyldiallylamine or of dialkyldiallylammonium, such as the homopolymers or copolymers comprising units corresponding to formula (VI) or (VI′):

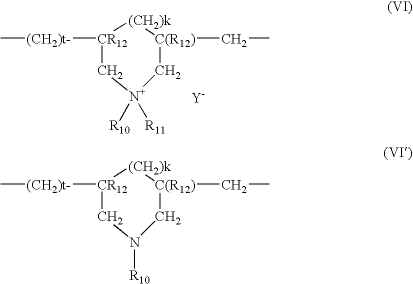

wherein formulae k and t are equal to 0 or 1, the sum k+t being equal to 1; R₁₂ is chosen from a hydrogen atom and a methyl group; R₁₀ and R₁₁, independently of each other, are chosen from an alkyl group comprising from 1 to 6 carbon atoms, a hydroxyalkyl group wherein the alkyl group comprises 1 to 5 carbon atoms, a lower amidoalkyl group, or R₁₀ and R₁₁ may form, together with the nitrogen atom to which they are attached, heterocyclic groups such as piperidyl or morpholinyl; Y⁻ is an anion such as bromide, chloride, acetate, borate, citrate, tartrate, bisulfate, bisulfite, sulfate or phosphate. These polymers are described, for example, in French Patent No. 2 080 759 and in its Certificate of Addition 2 190 406.

Additionally, non-limiting mention may be made, for example, of the diallyldimethylammonium chloride homopolymer sold under the name “Merquat® 100” by the company Ondeo-Nalco, and copolymers of diallyldimethylammonium chloride and of acrylamide.

(7) diquaternary ammonium polycondensates comprising repeating units corresponding to the formula:

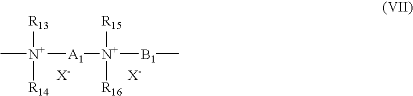

in which formula (VII):

R₁₃, R₁₄, R₁₅ and R₁₆, which may be identical or different, are chosen from aliphatic, alicyclic or arylaliphatic radicals comprising from 1 to 20 carbon atoms and lower hydroxyalkylaliphatic groups, or alternatively R₁₃, R₁₄, R₁₅ and R₁₆, together or separately, form, with the nitrogen atoms to which they are attached, heterocycles optionally comprising a second heteroatom other than nitrogen, or alternatively R₁₃, R₁₄, R₁₅ and R₁₆ are chosen from a linear and branched C₁-C₆ alkyl group substituted with a nitrile, ester, acyl or amide group or a group —CO—O—R₁₇—D or —CO—NH—R₁₇—D where R₁₇ is an alkylene group and D is a quaternary ammonium group;

A₁ and B₁ are chosen from linear and branched, saturated and unsaturated polymethylene groups comprising from 2 to 20 carbon atoms, which may comprise, linked to or intercalated in the main chain, at least one aromatic ring or at least one entity chosen from oxygen atoms, sulfur atoms, and sulfoxide, sulfbne, disulfide, amino, alkylamino, hydroxyl, quaternary ammonium, ureido, amide and ester groups, and

X⁻ is an anion derived from an inorganic or organic acid;

A₁, R₁₃ and R₁₅ may form, with the two nitrogen atoms to which they are attached, a piperazine ring; in addition, if A₁ is chosen from linear and branched, saturated and unsaturated alkylene and hydroxyalkylene groups, then B₁ may also be chosen from —(CH₂)ₙ—CO—D—OC—(CH₂)ₙ— groups in which D is chosen from:

a) glycol groups of formula: —O—Z—O—, where Z is chosen from a linear and branched hydrocarbon-based groups or groups of one of the following formulae:
—(CH₂—CH₂—O)ₓ—CH₂—CH₂—
—[CH₂—CH(CH₃)—O]_{y}—CH₂—CH(CH₃)—

where x and y are integers ranging from 1 to 4, representing a defined and unique degree of polymerization, or any number from 1 to 4 representing an average degree of polymerization;

b) bis-secondary diamine groups, such as a piperazine derivative;

c) bis-primary diamine groups of formula: —NH—Y—NH—, where Y is chosen from a linear and branched hydrocarbon-based groups, or alternatively the divalent radical —CH₂—CH₂—S—S—CH₂—CH₂—; and

d) ureylene ygroups of formula: —NH—CO—NH—;

For example, X⁻ may be an anion such as chloride or bromide.

These polymers may have a number-average molecular mass ranging from 1 000 to 100 000.

Polymers of this type are described for instance in French Patent Nos. 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945, and 4,027,020.

It is also possible, for example, to use polymers that comprise of repeating units of formula (a):

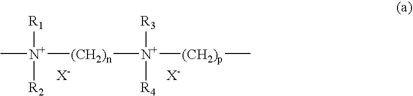

in which R₁, R₂, R₃ and R₄, which may be identical or different, are chosen from alkyl and hydroxyalkyl groups comprising from 1 to 4 carbon atoms, n and p are integers ranging from 2 to 20, and X⁻ is an anion derived from an inorganic or organic acid.

For example, in one embodiment, in the compound of formula (a), R₁, R₂, R₃ and R₄ are methyl groups, n is equal to 3, p is equal to 6 and X is a Cl atom, this compound is known as Hexadimethrine chloride according to the INCI (CTFA) nomenclature.

(8) polyquaternary ammonium polycondensates comprising of units of formula (VIII):

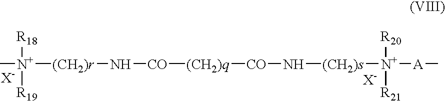

in which formula:

R₁₈, R₁₉, R₂₀ and R₂₁, which may be identical or different, are chosen from hydrogen atoms and methyl, ethyl, propyl, β-hydroxyethyl, β-hydroxypropyl and —CH₂CH₂(OCH₂CH₂)ₚOH groups,

p is an integer ranging from 0 to 6, with the proviso that R₁₈, R₁₉, R₂₀ and R₂₁, are not simultaneously all hydrogen atoms,

r and s, which may be identical or different, are integers ranging from 1 to 6,

q is an integer ranging from 0 to 34,

X is a halogen atom,

A is chosen from dihalide groups and —CH₂—CH₂—O—CH₂—CH₂— groups.

Non-limiting examples of such compounds are described in European Patent Application No. 122 324.

Among these products, non-limiting mention may be made, for example, of the products “Mirapol® A 15”, “Mirapol® AD1”, “Mirapol® AZ1” and “Mirapol® 175” sold by the company Miranol.

(9) homopolymers or copolymers derived from acrylic or methacrylic acids and comprising units:

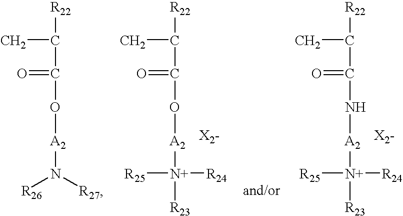

in which:

the groups R₂₂ are chosen from H atoms and CH₃ groups,

the groups A₂ are chosen from linear or branched alkyl groups of 1 to 6 carbon atoms and hydroxyalkyl groups of 1 to 4 carbon atoms,

the groups R₂₃, R₂₄ and R₂₅, which may be identical or different, are chosen from alkyl groups of 1 to 18 carbon atoms, and benzyl groups,

the group R₂₆ and R₂₇ are chosen from hydrogen atoms and alkyl groups of 1 to 6 carbon atoms,

X₂^{—} are chosen from anions, for example methosulfate and halides such as chloride or bromide.

The comonomer(s) that may be used in the preparation of the corresponding copolymers belong(s) to the family of acrylamides, methacrylamides, diacetone acrylamides, acrylamides and methacrylamides substituted on the nitrogen with lower alkyls, alkyl esters, acrylic and methacrylic acids, vinylpyrrolidone or vinyl esters.

(10) Quaternary polymers of vinylpyrrolidone and of vinylimidazole.

(11) Crosslinked methacryloyloxy(C₁-C₄)alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound comprising olefinic unsaturation, such as methylenebisacrylamide. Non-limiting mention may be made, for example, of Polyquaternium-37 (crosslinked methacryloyloxyethyltrimethylammonium chloride homopolymer) sold under the names Salcare® SC 95 and SC 96 by the company Ciba.

Other cationic polymers that can be used in the context of the present disclosure include polyalkyleneimines, such as polyethyleneimines, polymers comprising vinylpyridine or vinylpyridinium units, condensates of polyamines and of epichlorohydrin, quaternary polyureylenes and chitin derivatives.

Among all the cationic polymers that may be used in the context of the present disclosure, further non-limiting examples may be made of dialkyldiallylammonium halide homopolymers and copolymers, polyethyleneimines, polycondensates comprising diquaternary ammonium or polyquaternary ammonium repeating units, and crosslinked polymers of methacryloyloxy(C₁-C₄)alkyltri(C₁-C₄)alkylammonium salts.

The cationic polymers as disclosed herein are present in an amount ranging from 0.01% to 10% by weight, for example ranging from 0.05% to 5% by weight and further, for example, ranging from 0.1% to 3% by weight relative to the total weight of the composition.

The non-silicone solid fatty substances as disclosed herein may be crystalline, amorphous or pasty.

The melting point may range from 35 to 250° C. and for instance from 40 to 150° C.

These solids have a viscosity, at a temperature of 40° C. and at a shear rate of 1 s⁻¹, ranging from 1 Pa·s to 1 000 000 Pa·s, such as from 10 to 1000 Pa·s.

The viscosity measurements may be taken at a temperature of about 40° C., using a Carri-Med CSL2-500 viscometer.

The melting point may be measured by DSC or on a Kofler bench. The melting point may be measured by differential calorimetric analysis (DSC) with a temperature increase rate of 10° C./minute. The melting point is then the temperature corresponding to the top of the endothermic melting peak obtained during the measurement.

The non-silicone solid fatty substances with a melting point of greater than or equal to 35° C. may be chosen from oxyethylenated or non-oxyethylenated fatty alcohols, fatty esters, mineral waxes and organic waxes other than fatty esters and fatty alcohols, and mixtures thereof.

The fatty alcohols as disclosed herein may be linear and saturated, and comprise from 12 to 40 carbon atoms.

The fatty alcohols may have the structure R-OH, wherein R is chosen from C₁₂-C₂₄ alkyl groups. R may be substituted with at least one hydroxyl groups.

Non-limiting examples that may be mentioned include myristyl alcohol, cetyl alcohol, stearyl alcohol and behenyl alcohol, and mixtures thereof.

The fatty alcohol may represent a mixture of fatty alcohols, which means that, in a commercial product, several fatty alcohol species may coexist in the form of a mixture.

The fatty alcohols of the present disclosure may be non-oxyalkylenated and/or non-glycerolated. These fatty alcohols may be constituents of animal or plant waxes.

The fatty esters may be fatty acid esters, i.e., esters of a carboxylic acid comprising at least 10 carbon atoms and of a monoalcohol or a polyol. The fatty esters disclosed herein may be monoesters, diesters or triesters.

The carboxylic acids may comprise from 10 to 30 carbon atoms and for example from 12 to 24 carbon atoms. The alcohols may comprise from 10 to 30 carbon atoms and for example from 12 to 24 carbon atoms. The solid fatty esters as disclosed herein can be esters of a monocarboxylic fatty acid comprising at least 10 carbon atoms and of a monoalcohol comprising at least 10 carbon atoms.

Non-limiting mention of the esters, as disclosed herein, include cetyl myristate, myristyl myristate, palmityl palmitate, stearyl palmitate, palmityl stearate and stearyl stearate, and mixtures thereof.

The fatty esters may be constituents of animal or plant waxes.

For the purposes of the present disclosure, a wax is understood to mean a lipophilic compound that is solid at room temperature (about 25° C.), with a reversible solid/liquid change of state, having a melting point of greater than about 40° C., which may be up to 200° C., and having anisotropic crystal organization in the solid state. The size of the wax crystals is such that the crystals diffract and/or scatter light, giving the composition comprising them a more or less opaque hazy appearance. By bringing the wax to its melting point, it is possible to make it miscible with oils and to form a microscopically uniform mixture, but on returning the temperature of the mixture to room temperature, recrystallization of the wax in the oils of the mixture is obtained, which is detectable microscopically and macroscopically (opalescence).

As waxes that may be used in the present disclosure, non-limiting mention may be made of waxes of plant origin such as beeswax, spermaceti, lanolin wax and lanolin derivatives; plant waxes such as carnauba wax, candelilla wax, ouricury wax, Japan wax, cocoa butter wax, cork fibre wax or sugarcane wax; mineral waxes, for example paraffin wax, petroleum jelly wax, lignite wax or microcrystalline waxes, ozokerites, olive wax, rice wax, hydrogenated jojoba wax or the absolute waxes of flowers, such as the essential wax of blackcurrant blossom sold by the company Bertin (France), animal waxes, for instance beeswaxes, or modified beeswaxes (cerabellina); other waxes or waxy starting materials that may be used are marine waxes such as the product sold by the company Sophim under the reference M82, and mixtures thereof. Organic waxes that may also be mentioned include waxes comprising amide functions, such as natural or synthetic ceramides.

As used herein, the definition of waxes includes the meaning disclosed in P. D. Dorgan, Drug and Cosmetic Industry, December 1983, pp. 30-33.

The wax(es) may be chosen, in at least one embodiment, from carnauba wax, candelilla wax, esparto grass wax, paraffin wax, ozokerite, plant waxes, for instance olive wax, rice wax, hydrogenated jojoba wax or the absolute waxes of flowers, such as the essential wax of blackcurrant blossom sold by the company Bertin (France), animal waxes, for instance beeswaxes, or modified beeswaxes (cerabellina); other waxes or waxy starting materials that may be used are marine waxes, such as the product sold by the company Sophim under the reference M82.

The non-silicone solid fatty substance(s) may be present in the composition in an amount ranging from 0.1% to 10%, such as from 0.5% to 5% and further from 1% to 4% by weight relative to the total weight of the composition.

The composition, as disclosed herein, may optionally comprise surfactants.

The surfactants may be present in an amount ranging from 0.1% to 10%, for example, from 0.5% to 8% and further, for example, from 1% to 5% by weight, relative to the total weight of the composition.

The additional surfactants may be chosen from nonionic and cationic surfactants.

Nonionic surfactants are compounds that are known per se (see for example in this respect “Handbook of Surfactants” by M. R. Porter, published by Blackie & Son (Glasgow and London), 1991, pp. 116-178) and, in the context of the present disclosure, their nature is not a critical feature. Thus, among the nonionic surfactants that may be used, non-limiting mention may be made of polyethoxylated, polypropoxylated or polyglycerolated fatty alcohols, polyethoxylated, polypropoxylated, or polyglycerolated fatty α-diols, polyethoxylated, polypropoxylated or polyglycerolated fatty alkylphenols, or polyethoxylated, polypropoxylated or polyglycerolated fatty acids, all these compounds having a fatty chain comprising, for example, 8 to 18 carbon atoms, it being possible for the number of ethylene oxide or propylene oxide groups to ranging, for instance, from 2 to 50 and for the number of glycerol groups ranging, for example, from 2 to 30. Non-limiting mention may also be made of copolymers of ethylene oxide and of propylene oxide, condensates of ethylene oxide and of propylene oxide with fatty alcohols; polyethoxylated fatty amides comprising, for example, from 2 to 30 mol of ethylene oxide, polyglycerolated fatty amides comprising on average 1 to 5, such as 1.5 to 4, glycerol groups; oxyethylenated fatty acid esters of sorbitan comprising from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides or N-acylaminopropylmorpholine oxides.

The composition, as disclosed herein, comprises at least one cationic surfactant that is known per se, such as optionally polyoxyalkylenated primary, secondary or tertiary fatty amine salts and quaternary ammonium salts, and mixtures thereof.

In at least one embodiment of the present disclosure, the cationic surfactants are non-polymeric.

Non-limiting mention may be made of fatty amines that include alkylamidoamines, for instance (C₈-C₃₀)alkylamidodi(C₁-C₆)alkylamines and in particular stearamidopropyldimethylamine (MACKINE 301 sold by Maclntyre).

Non-limiting examples of quaternary ammonium salts that may be mentioned include:
those of formula (V) below: 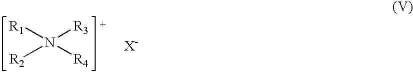
in which R₁ to R₄, which may be identical or different, are chosen from linear or branched aliphatic radicals comprising from 1 to 30 carbon atoms, and aromatic radicals such as aryl or alkylaryl. The aliphatic radicals may comprise heteroatoms such as oxygen, nitrogen, and sulfur, and halogens. The aliphatic radicals may be chosen, for example, from (C₁-C₃₀)alkyl, alkoxy, C₂-C₆ polyoxyalkylene, alkylamide, (C₁₂-C₂₂)alkylamido(C₂-C₆)alkyl, (C₁₂-C₂₂)alkylacetate and hydroxyalkyl radicals, comprising from about 1 to 30 carbon atoms; X⁻ is an anion chosen from halides, phosphates, acetates, lactates, (C₂-C₆) alkyl sulfates and alkyl- or alkylaryl-sulfonates;
quaternary ammonium salts of imidazoline, for instance those of formula (VI) below: 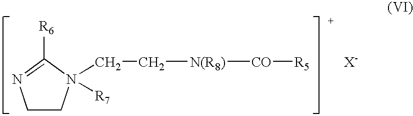
in which R₅ is an alkenyl or alkyl radical comprising from 8 to 30 carbon atoms, for example fatty acid derivatives of tallow or of coconut, R₆ is a hydrogen atom, a C₁-C₄ alkyl radical or an alkenyl or alkyl radical comprising from 8 to 30 carbon atoms, R₇ is a C₁-C₄ alkyl radical, R₈ is chosen from a hydrogen atom and a C₁-C₄ alkyl radical, and X is an anion chosen from halides, phosphates, acetates, lactates, alkyl sulfates, alkyl sulfonates or alkylaryl sulfonates. In at least one embodiment, R₅ and R₆ are chosen from a mixture of alkenyl or alkyl radicals comprising from 12 to 21 carbon atoms, such as fatty acid derivatives of tallow, R₇ is methyl and R₈ is a hydrogen atom. Such a product may be Quaternium-27 (CTFA 2002), Quaternium-87 (CTFA 2002) or Quaternium-83 (CTFA 2002), which are sold under the name “Varisoft®”W575PG by the company Goldschmidt,
diquaternary ammonium salts of formula (VII): 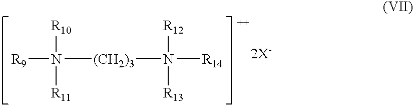
wherein R₉ is chosen from an aliphatic radical comprising from about 16 to 30 carbon atoms, R₁₀, R₁₁, R₁₂, R₁₃ and R₁₄, which may be identical or different, are chosen from hydrogen and an alkyl radical comprising from 1 to 4 carbon atoms, and X⁻ is an anion chosen from the group of halides, acetates, phosphates, nitrates, ethyl sulfates and methyl sulfates. Such diquaternary ammonium salts may comprise propanet allow diammonium dichloride;
quaternary ammonium salts comprising at least one ester function, such as those of formula (VIII) below: 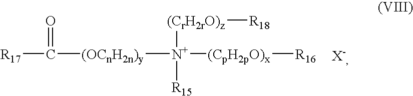
in which:
R₁₅ is chosen from C₁-C₆ alkyl radicals,C₁-C₆ hydroxyalkyl radicals, and C₁-C₆ dihydroxyalkyl radicals;
R₁₆ is chosen from: 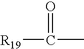
a radical
linear or branched, saturated or unsaturated C₁-C₂₂ hydrocarbon-based radicals R₂₀,
a hydrogen atom,
R₁₇ is chosen from: 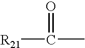
a radical
linear or branched, saturated or unsaturated C₁-C₆ hydrocarbon-based radicals R₂₂,
a hydrogen atom,
R₁₇, R₁₉ and R₂₁, which may be identical or different, are chosen from linear or branched, saturated or unsaturated C₇-C₂₁ hydrocarbon-based radicals;
r, n and p, which may be identical or different, are integers ranging from 2 to 6;
y is an integer ranging from 1 to 10;
x and z, which may be identical or different, are integers ranging from 0 to 10;
X⁻ is a simple or complex, organic or inorganic anion;
with the proviso that the sum x+y+z is from 1 to 15, that when x is 0, then R₁₆ is R₂₀ and that when z is 0, then R₁₈ is R₂₂.

The alkyl radicals R₁₅ may be linear or branched, and in at least one embodiment are linear.

Additionally, R₁₅ may be chosen from methyl, ethyl, hydroxyethyl and dihydroxypropyl radicals. In at least one embodiment, R₁₅ is chosen from methyl and ethyl radicals.

In at least one embodiment, the sum x+y+z is from 1 to 10.

When R₁₆ is a hydrocarbon-based radical R₂₀, it may be long and comprise from 12 to 22 carbon atoms, or short and comprise from 1 to 3 carbon atoms.

When R₁₈ is a hydrocarbon-based radical R₂₂, it comprises 1 to 3 carbon atoms.

R₁₇, R₁₉ and R₂₁, which may be identical or different, are chosen from linear or branched, saturated or unsaturated C₁₁-C₂₁ hydrocarbon-based radicals, such as from linear or branched, saturated or unsaturated C₁₁-C₂₁ alkyl and alkenyl radicals.

In at least one embodiment, x and z, which may be identical or different, are 0 or 1.

In at least one embodiment, y is equal to 1.

In at least one embodiment, r, n and p, which may be identical or different, are equal to 2 or 3, and in at least one further embodiment, equal to 2.

The anion X⁻ may be a halide (chloride, bromide or iodide) or a C₁-C₄ alkyl sulfate, such as methyl sulfate. However, methanesulfonate, phosphate, nitrate, tosylate, an anion derived from an organic acid, such as acetate or lactate, or any other anion that is compatible with the ammonium comprising an ester function may be used. In at least one embodiment, the anion X⁻ is chosen from chloride and methyl sulfate.

In one embodiment of the present disclosure, the composition, as disclosed herein, may be a conditioner comprising ammonium salts of formula (IV) in which:
R₁₅ is chosen from methyl and ethyl radicals,
x and y are equal to 1;
z is equal to 0 or 1;
r, n and p are equal to 2;
R₁₆ is chosen from:
a radical 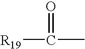
methyl, ethyl or C₁₄-C₂₂ hydrocarbon-based radicals,
a hydrogen atom;
R₁₈ is chosen from:
a radical R 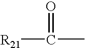
a hydrogen atom;
R₁₇, R₁₉ and R₂₁, which may be identical or different, are chosen from linear or branched, saturated or unsaturated C₁₃-C₁₇ hydrocarbon-based radicals, such as from linear or branched, saturated or unsaturated C₁₃-C₁₇ alkyl and alkenyl radicals. In at least one embodiment, the hydrocarbon-based radicals are linear.

Non-limiting examples of compounds of formula (VIII) that may be mentioned include the salts (such as chloride or methyl sulfate) of diacyloxyethyl-dimethylammonium, of diacyloxyethyl-hydroxyethyl-methylammonium, of monoacyloxyethyl-dihydroxyethyl-methylammonium, of triacyloxyethyl-methylammonium, of monoacyloxyethyl-hydroxyethyl-dimethylammonium, and mixtures thereof. The acyl radicals may comprise 14 to 18 carbon atoms and may be derived from a plant oil, for instance palm oil or sunflower oil. When the compound comprise several acyl radicals, these radicals may be identical or different.

These products are obtained, for example, by direct esterification of optionally oxyalkylenated triethanolamine, triisopropanolamine, alkyldiethanolamine or alkyldiisopropanolamine onto fatty acids or onto mixtures of fatty acids of plant or animal origin, or by transesterification of the methyl esters thereof. This esterification is followed by a quaternization using an alkylating agent such as an alkyl halide (e.g., a methyl or ethyl halide), a dialkyl sulfate (such as dimethyl or diethyl sulfate), methyl methanesulfonate, methyl para-toluenesulfonate, glycol chlorohydrin or glycerol chlorohydrin.

Such compounds are sold, for example, under the names Dehyquart® by the company Cognis, Stepanquat® by the company Stepan, Noxamium® by the company Ceca, and Rewoquat® WE 18 by the company Rewo-Goldschmidt.

The composition, as disclosed herein, may comprise a mixture of quaternary ammonium mono-, di- and triester salts with a weight majority of diester salts.

Non-limiting examples of mixtures of ammonium salts that may be used include the mixture comprising 15% to 30% by weight of acyloxyethyl-dihydroxyethyl-methylammonium methyl sulfate, 45% to 60% of diacyloxyethyl-hydroxyethyl-methylammonium methyl sulfate and 15% to 30% of triacyloxyethyl-methylammonium methyl sulfate, the acyl radicals comprising from 14 to 18 carbon atoms and being derived from optionally partially hydrogenated palm oil.

It is also possible to use the ammonium salts comprising at least one ester function described in U.S. Pat. Nos. 4,874,554 and 4,137,180.

Among the quaternary ammonium salts mentioned above, those corresponding to the above formula (V) may be used in at least one embodiment. Non-limiting mention of the quaternary ammonium salts may include tetraalkylammonium chlorides, for instance dialkyldimethylammonium or alkyltrimethylammonium chlorides in which the alkyl radical comprise from about 12 to 22 carbon atoms, such as behenyltrimethylammonium, distearyldimethylammonium, cetyltrimethylammonium or benzyldimethylstearylammonium chlorides, or alternatively palmitylamidopropyl-trimethylammonium chloride or stearamidopropyldimethyl(myristyl acetate)ammonium chloride corresponding to Quaternium-70 (CTFA 2002) sold under the name Ceraphyl® 70 by the company ISP.

The cationic surfactants that may be used in the composition of the disclosure are chosen from quaternary ammonium salts and, for example, from behenyltrimethylammonium chloride, cetyltrimethylammonium chloride, Quaternium-83, Quaternium-87, behenylamidopropyl-2,3-dihydroxypropyldimethylammonium chloride and palmitylamidopropyltrimethylammonium chloride.

When they are present, the cationic surfactant(s) are present in the presently disclosed composition in an amount ranging from 0.05% to 10% by weight, such as from 0.1% to 8% by weight and, for example, from 0.2% to 5% by weight relative to the total weight of the composition.

The compositions according to the present disclosure may be non-washing (non-detergent) compositions comprising, in at least one embodiment, less than 4% by weight and, for example, less than 1% by weight of anionic surfactants relative to the total weight of the composition.

The composition may also comprise at least one additional conditioning agent chosen from silicones, cationic polymers other than the cationic polymers according to the present disclosure, carboxylic fatty esters other than those of the disclosure, plant oils, mineral oils and synthetic oils such as poly(α-olefins), and mixtures thereof.

In another embodiment of the present disclosure, the silicones may be soluble or insoluble in the composition, and they may polyorganosiloxanes that are insoluble in the composition of the disclosure. They may be in the form of oils, waxes, resins or gums. They may be used pure or as an emulsion, a dispersion or a microemulsion.

The organopolysiloxanes are defined in greater detail in Walter Noll's “Chemistry and Technology of Silicones” (1968) Academic Press. They can be volatile or non-volatile.

When they are volatile, the silicones may be chosen, in at least one embodiment, from those having a boiling point ranging from 60° C. to 260° C., and from:

(i) cyclic silicones comprising from 3 to 7 and for instance from 4 to 5 silicon atoms. These are, for example, octamethylcyclotetrasiloxane sold in particular under the name “Volatile Silicone 7207” by Union Carbide or “Silbione 70045 V 2” by Rhodia, decamethylcyclopentasiloxane sold under the name “Volatile Silicone 7158” by Union Carbide, and “Silbione 70045 V 5” by Rhodia, and mixtures thereof.

Non-limiting mention may also be made of cyclocopolymers of the dimethylsiloxane/methylalkylsiloxane type, such as “Silicone Volatile FZ 3109” sold by the company Union Carbide, having the chemical structure:

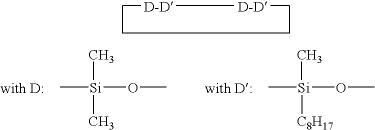

Non-limiting mention may also be made of mixtures of cyclic silicones with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;

(ii) linear volatile silicones comprising 2 to 9 silicon atoms and having a viscosity of less than or equal to 5×10⁻⁶ m²/s at 25° C. An example is decamethyltetrasiloxane sold under the name “SH 200” by the company Toray Silicone. Silicones belonging to this category are also described in the article published in Cosmetics and Toiletries, Vol. 91, January 76, pp. 27-32, Todd & Byers “Volatile Silicone Fluids for Cosmetics”.

Non-limiting examples of the non-volatile silicones that may be mentioned include polyalkylsiloxanes, polyarylsiloxanes, polyalkylarylsiloxanes, silicone gums and resins, polyorganosiloxanes modified with organofunctional groups, and also mixtures thereof.

In yet another embodiment of the present disclosure, the organomodified silicones that can be used are silicones as defined above and comprising in their structure at least one organofunctional group attached via a hydrocarbon-based group.

Among the organomodified silicones, non-limiting mention may be made of polyorganosiloxanes comprising:

polyethyleneoxy and/or polypropyleneoxy groups optionally comprising C₆-C₂₄ alkyl groups, such as the products known as dimethicone copolyol sold by the company Dow Corning under the name DC 1248 or the oils Silwet® L 722, L 7500, L 77 and L 711 by the company Union Carbide, and the (C₁₂)alkylmethicone copolyol sold by the company Dow Corning under the name Q2 5200;
substituted or unsubstituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups may be, for example, C₁—C₄ aminoalkyl groups;
thiol groups such as the products sold under the names “GP 72 A” and “GP 71” from Genesee;
alkoxylated groups such as the product sold under the name “Silicone Copolymer F-755” by SWS Silicones and Abil Wax® 2428, 2434 and 2440 by the company Goldschmidt;
hydroxylated groups such as the polyorganosiloxanes comprising a hydroxyalkyl function, described in French Patent No. 2,589,476;
acyloxyalkyl groups such as, for example, the polyorganosiloxanes described in U.S. Pat. No. 4,957,732;
anionic groups of the carboxylic acid type, such as, for example, in the products described in European Patent No. 186 507 from the company Chisso Corporation, or of the alkylcarboxylic type, such as those present in the product X-22-3701^{E} from the company Shin-Etsu; 2-hydroxyalkyl sulfonate; 2-hydroxyalkyl thiosulfate such as the products sold by the company Goldschmidt under the names “Abil® S201” and “Abil® S255”;
-hydroxyacylamino groups, such as the polyorganosiloxanes described in European Patent Application No. 342 834. Non-limiting mention may be made, for example, of the product Q2-8413 from the company Dow Corning.

Non-limiting examples of silicones that may be used include polydimethylsiloxanes, polyalkylarylsiloxanes and polydimethylsiloxanes comprise amino or alkoxylated groups.

The composition, as disclosed herein, may also comprise at least one liquid carboxylic acid ester, for instance purcellin oil (stearyl octanoate), isopropyl myristate, isopropyl palmitate, butyl stearate, hexyl laurate, diisopropyl adipate, isononyl isononanoate, 2-ethylhexyl palmitate, 2-octyldodecyl lactate, isostearyl neopentanoate, tridecyl neopentanoate, isocetyl neopentanoate and isoarachidyl neopentanoate, and mixtures thereof.

The composition, as disclosed herein, may also comprise at least one plant oil such as sweet almond oil, avocado oil, castor oil, olive oil, jojoba oil, sunflower oil, wheatgerm oil, sesame oil, groundnut oil, grapeseed oil, soybean oil, rapeseed oil, safflower oil, coconut oil, corn oil, hazelnut oil, Shea butter, palm oil, apricot kernel oil and beauty-leaf oil, and mixtures thereof.

Additional mineral oils that may also be used include, for example, liquid paraffin and liquid petroleum jelly.

The additional conditioning agents, in at least one embodiment, are present in the composition, as disclosed herein, in an amount ranging from 0.01% to 20% by weight, for example, ranging from 0.1% to 10% by weight and further, for example, ranging from 0.3% to 5% by weight relative to the total weight of the composition.

In at least one embodiment, the cosmetically acceptable medium is aqueous and may comprise water or a mixture of water and a cosmetically acceptable solvent such as a C₁-C₄ lower alcohol, for example ethanol, isopropanol, tert-butanol or n-butanol; polyols, for instance propylene glycol or glycerol; polyol ethers; C₅-C₁₀ alkanes; and mixtures thereof. In a further embodiment, the solvents are chosen from glycerol and propylene glycol.

The cosmetically acceptable medium, which is aqueous in at least one embodiment, is present in an amount of 30% to 98% by weight relative to the total weight of the composition.

In at least one embodiment, the solvents are present in concentrations ranging from 0.5% to 30% by weight relative to the total weight of the composition.

The pH of the compositions of the present disclosure ranges from 2 to 8 and, for example, ranging from 3 to 7.

The compositions according to the present disclosure may also comprise standard additives that are known in the art, such as anionic, nonionic or amphoteric polymers, non-polymeric thickeners, for instance acids or electrolytes, opacifiers, nacreous agents, vitamins, provitamins such as panthenol, fragrances, dyes, organic or mineral particles, preserving agents and pH stabilizers.

A person skilled in the art will take care to select the optional additives and the amount thereof such that they do not harm the properties of the compositions of the present disclosure.

These additives are present in the composition according to the present disclosure in an amount ranging from 0% to 20% by weight relative to the total weight of the composition.

The compositions of the present disclosure may be in the form of a rinse-out or leave-in conditioner, permanent waving, relaxing, dyeing or bleaching compositions, or alternatively in the form of rinse-out compositions to be applied before or after a dyeing, bleaching, permanent-waving or relaxing operation or alternatively between the two steps of a permanent-waving or relaxing operation.

They may be used, for example, as hair conditioners, care products, deep-down care masks or scalp treatment lotions or creams. These compositions may be rinse-out or leave-in compositions.

In one embodiment of the present disclosure, the composition may be used as a hair conditioner, for example on sensitized hair. This hair conditioner may be a rinse-out or leave-in hair conditioner, for instance a rinse-out hair conditioner.

The cosmetic compositions according to the present disclosure may be in the form of a gel, a milk, a cream, an emulsion, fluid or thickened lotions or a foam, and may be used for the skin, the nails, the eyelashes, the lips and the hair.

The compositions may be packaged in various forms, such as in vaporizers, pump-dispenser bottles or in aerosol containers in order to dispense the composition in vaporized form or in the form of a mousse. Such packaging forms are indicated, for example, when it is desired to obtain a spray, a lacquer or a mousse for treating the hair.

The present disclosure also relates to a cosmetic process for treating keratin materials such as, for example, the skin or the hair, which consists in applying an effective amount of a cosmetic composition, as described above, to the keratin materials, and optionally rinsing it off after optionally leaving it to act for a period of time.

The rinsing is performed, for example, with water.

Thus, this process, as disclosed herein, allows the treatment, conditioning and care of the hair or any other keratin material.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

The following examples are intended to illustrate the invention in a non-limiting manner.

### EXAMPLES 1 TO 8

The following rinse-out hair-conditioning compositions were prepared:
1. All the water was introduced into the manufacturing tank, and the water-soluble compounds were added, with the exception of the oxyethylenated sorbitan ester. The mixture was heated to 80° C. with stirring using a doctor blade, until fully dissolved.
2. The lipophilic compounds were introduced apart from the silicones, if any, and the fragrances into an additional tank, and heated to 80° C.
3. The contents of the additional tank and the silicones, if any, were introduced into the manufacturing tank and emulsified for 10 minutes with vigorous turbomixer and doctor blade stirring, while maintaining the temperature. Then, cooling was begun.
4. At 30° C., the oxyethylenated sorbitan ester and the fragrance were introduced with doctor blade stirring.

### Example 1

| | in g AM |
|---|---|
| Copolymer of SMDI (saturated methylene diphenyl | 0.45 |
| diisocyanate)/polyethylene glycol containing alkyl end groups | |
| (methyl/C18) at 15% AM | |
| (ACULYN 46 from Rohm & Haas) | |
| Crosslinked ethyltrimethylammonium methacrylate chloride | 0.55 |
| homopolymer at 50% AM | |
| (SALCARE SC 95 from Ciba) | |
| Cetylstearyl alcohol | 2.5 |
| (LANETTE O OR from Cognis) | |
| Sorbitan monolaurate oxyethylenated with 4 mol of ethylene | 4 |
| oxide (4 EO) | |
| (TWEEN 21 from Uniqema) | |
| Fragrance | qs |
| Preserving agents | qs |
| Water qs | 100 g |

### Example 2

| | in g AM |
|---|---|
| Copolymer of SMDI (saturated methylene diphenyl | 0.6 |
| diisocyanate)/polyethylene glycol containing alkyl end groups | |
| (methyl/C18) at 15% AM | |
| (ACULYN 46 from Rohm & Haas) | |
| Crosslinked ethyltrimethylammonium methacrylate chloride | 0.55 |
| homopolymer at 50% AM | |
| (SALCARE SC 95 from Ciba) | |
| Cetylstearyl alcohol | 2 |
| (LANETTE O OR from Cognis) | |
| Sorbitan monolaurate oxyethylenated with 4 mol of ethylene | 4 |
| oxide (4 EO) | |
| (TWEEN 21 from Uniqema) | |
| Piroctone olamine | 0.3 |
| (OCTOPIROX from Clariant) | |
| Fragrance | qs |
| Preserving agents | qs |
| Water qs | 100 g |

### Example 3

| | in g AM |
|---|---|
| Copolymer of SMDI (saturated methylene diphenyl | 0.45 |
| diisocyanate)/polyethylene glycol containing alkyl end groups | |
| (methyl/C18) at 15% AM | |
| (ACULYN 46 from Rohm & Haas) | |
| Crosslinked ethyltrimethylammonium methacrylate chloride | 0.55 |
| homopolymer at 50% AM | |
| (SALCARE SC 95 from Ciba) | |
| Cetylstearyl alcohol | 2 |
| (LANETTE O OR from Cognis) | |
| Sorbitan monolaurate oxyethylenated with 4 mol of ethylene | 4 |
| oxide (4 EO) | |
| (TWEEN 21 from Uniqema) | |
| Aminexil | 0.3 |
| Fragrance | qs |
| Preserving agents | qs |
| Water qs | 100 g |

### Example 4

| | in g AM |
|---|---|
| Copolymer of SMDI (saturated methylene diphenyl | 0.45 |
| diisocyanate)/polyethylene glycol containing alkyl end groups | |
| (methyl/C18) at 15% AM | |
| (ACULYN 46 from Rohm & Haas) | |
| Crosslinked ethyltrimethylammonium methacrylate chloride | 0.55 |
| homopolymer at 50% AM | |
| (SALCARE SC 95 from Ciba) | |
| Cetylstearyl alcohol | 2 |
| (LANETTE O OR from Cognis) | |
| Sorbitan monolaurate oxyethylenated with 20 mol of ethylene | 4 |
| oxide (20 EO) | |
| (TWEEN 20 from Uniqema) | |
| Diaminopyrimidine oxide | 1.5 |
| (MEXORYL SAG from Chimex) | |
| Fragrance | qs |
| Preserving agents | qs |
| Water qs | 100 g |

### Example 5

### Rinse-Out Hair Conditioner

| | in g AM |
|---|---|
| Copolymer of SMDI (saturated methylene diphenyl | 0.45 |
| diisocyanate)/polyethylene glycol containing alkyl end groups | |
| (methyl/C18) at 15% AM | |
| (ACULYN 46 from Rohm & Haas) | |
| Polydimethyldiallylammonium chloride at 40% AM | 0.4 |
| (MERQUAT 100 from Nalco) | |
| Cetylstearyl alcohol | 2 |
| (LANETTE O OR from Cognis) | |
| Sorbitan monolaurate oxyethylenated with 20 mol of ethylene | 4 |
| oxide (20 EO) | |
| (TWEEN 20 from Uniqema) | |
| Fragrance | qs |
| Preserving agents | qs |
| Water qs | 100 g |

### Example 6

### Rinse-Out Hair Conditioner

| | in g AM |
|---|---|
| Copolymer of SMDI (saturated methylene diphenyl | 0.45 |
| diisocyanate)/polyethylene glycol containing alkyl end groups | |
| (methyl/C18) at 15% AM | |
| (ACULYN 46 from Rohm & Haas) | |
| Polydimethyldiallylammonium chloride at 40% AM | 0.4 |
| (MERQUAT 100 from Nalco) | |
| Cetylstearyl alcohol | 2 |
| (LANETTE O OR from Cognis) | |
| Sorbitan monolaurate oxyethylenated with 4 mol of ethylene | 4 |
| oxide (4 EO) | |
| (TWEEN 21 from Uniqema) | |
| Fragrance | qs |
| Preserving agents | qs |
| Water qs | 100 g |

### Example 7 Rinse-Out Hair Conditioner

| | in g AM |
|---|---|
| Candelilla wax | 0.4 |
| Stearyl alcohol | 2 |
| (LANETTE 18 from Cognis) | |
| Behenyltrimethylammonium chloride at 80% AM | 0.5 |
| (GENAMINE KDMP from Clariant) | |
| Tetramethylhexamethylenediamine polycondensate as an | 0.4 |
| aqueous solution containing 60% AM | |
| (MEXOMER PO from Chimex) | |
| Hydroxypropylated and pregelatinized corn distarch phosphate | 1.5 |
| (STRUCTURE ZEA from National Starch) | |
| Sorbitan monolaurate oxyethylenated with 4 mol of ethylene | 4 |
| oxide (4 EO) | |
| (TWEEN 21 from Uniqema) | |
| Fragrance | qs |
| Preserving agents | qs |
| Water qs | 100 g |

### Example 8

| | in g AM |
|---|---|
| Copolymer of SMDI (saturated methylene diphenyl | 0.45 |
| diisocyanate)/polyethylene glycol containing alkyl end groups | |
| (methyl/C18) at 15% AM | |
| (ACULYN 46 from Rohm & Haas) | |
| Crosslinked ethyltrimethylammonium methacrylate chloride | 0.55 |
| homopolymer containing 50% AM | |
| (SALCARE SC 95 from Ciba) | |
| Cetylstearyl alcohol | 2.5 |
| (LANETTE O OR from Cognis) | |
| Sorbitan monostearate oxyethylenated with 20 EO | 2 |
| (TWEEN 60 from Uniqema) | |
| Fragrance | qs |
| Preserving agents | qs |
| Water qs | 100 g |

These compositions were applied to very sensitized hair. The cosmetic properties (disentangling, smoothness and suppleness) were excellent and uniform from the roots to the ends of the hair. The ends were not split.

Between two applications, the hair remained soft, supple and smooth.

What is claimed is:

1. A cosmetic composition comprising, in a cosmetically acceptable aqueous medium,
at least one ester of oxyethylenated sorbitan and of a saturated or unsaturated, linear or branched C₈-C₃₀ fatty acid, with a number of moles of ethylene oxide of less than or equal to 20,
at least one non-silicone cationic polymer with a cationic charge density of greater than or equal to 4 meq/g, and
at least one non-silicone solid fatty substance.

2. A composition according to claim 1 wherein said fatty acid comprises from 8 to 24 carbon atoms.

3. A composition according to claim 2, wherein said fatty acid comprises from 8 to 18 carbon atoms.

4. A composition according to claim 2 wherein said fatty acid is chosen from lauric acid and stearic acid.

5. A compositions according to claim 4, wherein said fatty acid is lauric acid.

6. A composition according to claim 1, wherein the at least one oxyethylenated sorbitan ester is chosen from monoesters of a C₈-C₂₄ fatty acid and of oxyethylenated sorbitan.

7. A composition according to claim 1, wherein the number of moles of ethylene oxide is less than 10 mol of ethylene oxide.

8. A composition according to claim 7, wherein the number of moles of ethylene oxide ranges from 3 to 8 mol of ethylene oxide.

9. A composition according to claim 8, wherein the number of moles of ethylene oxide is 4 mol of ethylene oxide.

10. A composition according to claim 1, wherein the least one oxyethylenated sorbitan ester is chosen from sorbitan monolaurate oxyethylenated with 4 mol of ethylene oxide (4 EO) and sorbitan monostearate oxyethylenated with 4 mol of ethylene oxide (4 EO).

11. A composition according to claim 10, wherein the at least one oxyethylenated sorbitan ester is sorbitan monolaurate oxyethylenated with 4 mol of ethylene oxide (4 EO).

12. A composition according to claim 1, wherein the at least one oxyethylenated sorbitan ester is present in the composition in an amount ranging from 0.1% to 10% by weight relative to the total weight of said composition.

13. A composition according to claim 12, wherein the at least one oxyethylenated sorbitan ester is present in the composition in an amount ranging from 0.5% to 5% by weight relative to the total weight of said composition.

14. A cosmetic composition according to claim 1, wherein the at least one non-silicone cationic polymer has a cationic charge density ranging from 4 to 25 meq/g.

15. A cosmetic composition according to claim 14, wherein the at least one non-silicone cationic polymer has a cationic charge density ranging from 4 to 8 meq/g.

16. A composition according to claim 1, wherein the at least one non-silicone cationic polymer is chosen from dialkyldiallylammonium halide homopolymers and copolymers, polycondensates comprising diquaternary or polyquaternary ammonium repeating units, polyethyleneimines, and crosslinked polymers of methacryloyloxy(C₁-C₄)alkyltri(C₁-C₄)alkylammonium salts.

17. A composition according to claim 1, wherein the at least one non-silicone cationic polymer is present at a concentration ranging from 0.01% to 10% by weight relative to the total weight of the composition.

18. A composition according to claim 17, wherein the at least one non-silicone cationic polymer is present at a concentration ranging from 0.05% to 5% by weight relative to the total weight of the composition.

19. A composition according to according to claim 1, wherein the at least one non-silicone solid fatty substance is chosen from oxyethylenated or non-oxyethylenated fatty alcohols, fatty esters, mineral waxes, and organic waxes other than the fatty esters and fatty alcohols, and mixtures thereof.

20. A composition according to claim 1, wherein the at least one non-silicone solid fatty substance is a plant or animal wax.

21. A composition according to claim 1, wherein the at least one non-silicone solid fatty substance is a linear fatty alcohol.

22. A composition according to claim 1, wherein the at least one non-silicone solid fatty substance is a saturated fatty alcohol.

23. A composition according to claim 1, wherein the at least one non-silicone solid fatty substance is a fatty alcohol having the structure R—OH, in which R is an optionally hydroxylated C₁₂-C₂₄ alkyl radical.

24. A composition according to claim 1, wherein the at least one non-silicone solid fatty substance is a fatty alcohol chosen from myristyl alcohol, cetyl alcohol, behenyl alcohol and stearyl alcohol, and mixtures thereof.

25. A composition according to claim 19, wherein the fatty esters comprise at least 10 carbon atoms and are the fatty esters of a carboxylic acid and of a monoalcohol or a polyol.

26. A composition according to claim 19, wherein the fatty esters are chosen from esters of a monocarboxylic fatty acid comprising at least 10 carbon atoms and of a monoalcohol comprising at least 10 carbon atoms.

27. A composition according to claim 19, wherein the fatty esters are chosen from cetyl myristate, myristyl myristate, palmityl palmitate, stearyl palmitate, palmityl stearate and stearyl stearate, and mixtures thereof.

28. A composition according to claim 19, wherein the waxes are chosen from beeswax, spermaceti, lanolin wax and lanolin derivatives; carnauba wax, candelilla wax, ouricury wax, Japan wax, cocoa butter, cork fibre wax or sugarcane wax; paraffin wax, petroleum jelly wax, lignite wax or microcrystalline waxes, ozokerite, olive wax, rice wax, hydrogenated jojoba wax, the absolute waxes of flowers, beeswaxes or modified beeswaxes (cerabellina), and natural or synthetic ceramides, and mixtures thereof.

29. A composition according to claim 1, wherein the at least one non-silicone solid fatty substance is present in the composition at a concentration ranging from 0.1% to 10% by weight relative to the total weight of the composition.

30. A composition according to claim 29, wherein the at least one non-silicone solid fatty substance is present in the composition at a concentration ranging from 0.5% to 5% by weight relative to the total weight of the composition.

31. A composition according to claim 1, further comprising at least one cationic surfactant.

32. A composition according to claim 31, wherein the at least one cationic surfactant is chosen from the salts of optionally polyoxyalkylenated primary, secondary or tertiary fatty amines and quaternary ammonium salts, and mixtures thereof.

33. A composition according to claim 31, wherein the at least one cationic surfactant is chosen from behenyltrimethylammonium chloride, cetyltrimethylammonium chloride, Quaternium-83, Quaternium-87, behenylamidopropyl 2,3-dihydroxypropyl dimethyl ammonium chloride and palmitylamidopropyltrimethylammonium chloride.

34. A composition according to claim 31, wherein the at least one cationic surfactant is present in an amount ranging from 0.05% to 10% by weight relative to the total weight of the composition.

35. A composition according to claim 34, wherein the at least one cationic surfactant is present in an amount ranging from 0.1% to 8% by weight relative to the total weight of the composition.

36. A composition according to claim 1, further comprising at least one additional conditioning agent.

37. A composition according to claim 36, wherein the at least one additional conditioning agent is chosen from silicones, cationic polymers other than the non-silicone cationic polymers, liquid carboxylic esters, plant oils, mineral oils and synthetic oils, and mixtures thereof.

38. A composition according to claim 1, wherein the cosmetically acceptable aqueous medium comprises water or a mixture of water and at least one cosmetically acceptable solvent.

39. A composition according to claim 38, wherein the cosmetically acceptable solvent is chosen from C₁-C₄ lower alcohols; polyol ethers; C₅-C₁₀ alkanes, and mixtures thereof.

40. A composition according to claim 1, further comprising at least one additive chosen from anionic, nonionic or amphoteric polymers, thickeners, opacifiers, nacreous agents, vitamins, provitamins, fragrances, dyes, organic or mineral particles, preserving agents and pH stabilizers.

41. A composition according to claim 1, wherein said composition is in a form chose from a hair conditioner, a composition for permanent-waving, relaxing, dyeing or bleaching the hair, and a rinse-out composition to be applied between the two steps of a permanent-waving or hair-relaxing operation.

42. A composition according to claim 1, wherein said composition is in the form of a rinse-out hair conditioner.

43. A cosmetic process for treating keratin fibers comprising
applying to said fibers an effective amount of a cosmetic composition comprising, in a cosmetically acceptable aqueous medium,
at least one ester of oxyethylenated sorbitan and of a saturated or unsaturated, linear or branched C₈-C₃₀ fatty acid, with a number of moles of ethylene oxide of less than or equal to 20,
at least one non—silicone cationic polymer with a cationic charge density of greater than or equal to 4 meq/g, and
at least one non-silicone solid fatty substance, and
optionally rinsing said keratin fibers.
